SEQUENCE LISTING (4 sequences)

SEQ ID NO: 1 - protein, 785 aa, Artificial
  HIStagged IFI16 protein
MHHHHHHSSGLVPRGSGMKETAAAKFERQHMDSPDLGTDDDDKAMADIGSSLMSVKMGKKYKNIVLLKGLEVINDYHFRMVKSLLSNDLKLNLKMREEYDKIQIADLMEEKFRGDAGLGKLIKIFEDIPTLEDLAETLKKEKLKVKGPALSRKRKKEVDATSPAPSTSSTVKTEGAEATPGAQKRKKSTKEKAGPKGSKVSEEQTQPPSPAGAGMSTAMGRSPSPKTSLSAPPNTSSTENPKTVAKCQVTPRRNVLQKRPVIVKVLSTTKPFEYETPEMEKKIMFHATVATQTQFFHVKVLNTSLKEKFNGKKIIIISDYLEYDSLLEVNEESTVSEAGPNQTFEVPNKIINRAKETLKIDILHKQASGNIVYGVFMLHKKTVNQKTTIYEIQDDRGKMDVVGTGQCHNIPCEEGDKLQLFCFRLRKKNQMSKLISEMHSFIQIKKKTNPRNNDPKSMKLPQEQRQLPYPSEASTTFPESHLRTPQMPPTTPSSSFFTKKSEDTISKMNDFMRMQILKEGSHFPGPFMTSIGPAESHPHTPQMPPSTPSSSFLTTLKPRLKTEPEEVSIEDSAQSDLKEVMVLNATESFVYEPKEQKKMFHATVATENEVFRVKVFNIDLKEKFTPKKIIAIANYVCRNGFLEVYPFTLVADVNADRNMEIPKGLIRSASVTPKINQLCSQTKGSFVNGVFEVHKKNVRGEFTYYEIQDNTGKMEVVVHGRLTTINCEEGDKLKLTCFELAPKSGNTGELRSVIHSHIKVIKTRKNKKDILNPDSSMETSPDFFF

SEQ ID NO: 2 - protein, 255 aa, Artificial [sequence omitted]

SEQ ID NO: 3 - protein, 255 aa, Artificial [sequence omitted]

SEQ ID NO: 4 - protein, 74 aa, Artificial
  HIStagged control peptide
MHHHHHHSSGLVPRGSGMKETAAAKFERQHMDSPDLGTDDDDKAMADIGSEFELRRQACGRTRAPPPPPLRSGC

(19) United States
(12) Patent Application Publication
(10) Pub. No.: US 2010/0105086 A1
(43) Pub. Date: Apr. 29, 2010

(54) DIFFERENTIAL DIAGNOSIS FOR SCLERODERMA

(76) Inventors: Santo Landolfo, Vercelli (IT); Michele Mondini, Vercelli (IT); Marisa Gariglio, Vercelli (IT)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/438,199

(22) PCT Filed: Aug. 20, 2007
(86) PCT No.: PCT/IB2007/002381

§ 371 Date: Jul. 16, 2009

(30) Foreign Application Priority Data
Aug. 21, 2006 (IT) TO2006A000614

(51) Int. Cl. G01N 33/535

(52) U.S. Cl. 435/7.92

(57) ABSTRACT

Use of IFI16 protein, fragments or peptides thereof for differential diagnosis of the limited cutaneous form of scleroderma (Ic-SSc) in a subject suspected of or at risk of having an autoimmune disease and the corresponding method of diagnosis and kit.

### TECHNICAL FIELD OF THE INVENTION

The present invention concerns the presence and clinical significance of autoantibodies directed against the interferon-inducible gene IFI16 in patients with systemic sclerosis/scleroderma (SSc), systemic lupus erythematosus (SLE) and other autoimmune diseases.

### BACKGROUND OF THE INVENTION

A family of interferon (IFN)-inducible genes, designated HIN200 in the human and Ifi200 in the murine species, encodes evolutionarily related human (IFI16, IFIX, MNDA, and AIM2) and murine proteins (p202, p203, p204, p205/D3) (1, 2). The IFI16 (Pubmed Accession No. NP_{—}005522), p202, and p204 nuclear phosphoproteins participate in the inhibition of cell cycle progression, modulation of differentiation, and cell survival. Gene expression analyses in congenic mice have identified p202 as a candidate gene for lupus susceptibility (3).

In 1994 Seelig et al. (4) reported the presence of anti-IFI16 antibodies in 29% of sera obtained from systemic lupus erythematosus (SLE) patients and recently Uchida et al. (5) in up to 70% of patients suffering from both primary and secondary Sjögren's syndrome (SjS). Lower prevalence has been reported in other systemic autoimmune diseases such as rheumatoid arthritis (RA) (0-13%) and scleroderma/polymyositis overlap syndrome (3%) (4, 5). However, the studies on SjS and RA enrolled small series of patients and no data are available on the presence of these autoantibodies in another frequent systemic autoimmune disease such as scleroderma (SSc).

Moreover, IFI16 expression in target tissues of the autoimmune process (i.e. the salivary glands) was described (5). This finding raises the possibility that a local tissue expression (or even up-regulation) can be pivotal in triggering an autoimmune response against this protein. Interestingly, physiological IFI16 expression was found in vascular endothelial cells and in stratified squamous epithelia such as skin (6). Both these tissues are targets for main SLE clinical manifestations (7). The present inventors looked at IFI16 expression in the lesional skin from SLE patients in order to investigate whether an enhanced IFI16 expression might be associated with the occurrence of autoantibodies directed against it. Since an increased serum levels of anti-IFI16 antibodies was also observed in SSc patients, the present inventors extended the study to SSc skin biopsies as well.

### SUMMARY OF THE INVENTION

The present invention relates to the expression of IFI16 in skin biopsy specimens from SSc and SLE patients. Levels of autoantibodies against IFI16 were determined by enzyme-linked immunosorbent assay (ELISA) in serum samples from 82 SSc and 100 SLE patients. Other autoimmune diseases (primary Sjögren's syndrome (SjS), rheumatoid arthritis (RA) and chronic urticaria (CU) were also examined.

The first aim of the present invention is to evaluate the presence and the titers of anti-IFI16 autoantibodies in a larger number of SjS, RA as well as in SSc patients and controls, and to partially characterize their antigenic specificity.

Object of the present invention is the provision of evidence that an IFN-inducible gene, IFI16, is involved in the pathophysiological mechanisms of connective tissue disorders such as SSc. More specifically, an object of the present invention is the provision of differential diagnosis methods for scleroderma, and, in particular, a novel tool in the differential diagnosis of lc-SSc (limited cutaneous form of scleroderma) from dc-SSc (diffused cutaneous form of scleroderma).

According to the present invention, said objects are achieved thanks to the solution having the characteristics referred to specifically in the ensuing claims. The claims form integral part of the technical teaching herein provided in relation to the present invention.

According to the present invention, said objects are achieved by means of the use of IFI16 protein, fragments or peptides thereof, wherein autoantibodies against IFI16 protein fragments or peptides thereof are detected. In a preferred embodiment, the diagnosis is based on the detection by an immunoassay (like for example ELISA, RIA, immunofluorescence or immunohistochemistry assay) of a higher level of autoantibodies against IFI16 protein, fragments or peptides thereof in the subject as compared to the normal level of these autoantibodies and/or to a level of these autoantibodies in patients suffering of a diffused cutaneous form of scleroderma (dc-SSc).

The differential diagnosis method according to a preferred embodiment of the present invention comprises:
a. obtaining a sample from a patient;
b. contacting the sample with an antigen comprising IFI16 protein, fragments or peptides thereof to form a complex of the autoantibody and the antigen;
c. detecting the presence of the complex autoantibody-antigen.

According to a preferred embodiment, the antigen of human, animal, synthetic or recombinant origin is bound to a solid carrier and the detection is performed using an anti-human IgG antibody, preferably conjugated to a detectable marker. The detectable marker is preferably selected from horseradish peroxidase, alkaline phosphatase, biotin or fluorescent dyes. The sample can be selected from whole blood, serum, plasma, saliva, tears, sweat, synovial fluid.

The present invention is also directed to a kit for the determination of anti-IFI16 autoantibodies in a sample preferably comprising a plate of wells having bound thereto IFI16 protein, fragments or peptides thereof and a detection reagent.

### DETAILED DESCRIPTION OF THE INVENTION

The present invention will now be described in detail in relation to some preferred embodiments by way of non-limiting examples.

### BRIEF DESCRIPTION OF THE DRAWINGS

FIG. 1. Increased expression of IFI16 in SLE and SSc lesions compared with skin from healthy controls. Expression of IFI16 in skin from healthy control (A), SLE lesion (B) and SSc lesion (C) (Original magnification×10). D, Enlarged section of derma from Panel C showing IFI16 staining in inflammatory cells (Original magnification ×20).

FIG. 2. A, IgG titers against human recombinant IFI16 in patients with SSc (82), SLE (100), primary SjS (20), RA (50), CU (38) and HCV infection (40) and from healthy controls (80). Boxes show values within 25^{th} and 75^{th} percentiles, the horizontal bar represents the median, 80% of values are between the extremities of the vertical bars (10^{th}-90^{th} percentiles), and extreme values are represented by individual symbols. Differences between the groups have been evaluated by one-way ANOVA and Bonferroni Multiple Comparisons tests after logarithmic transformation. Values under the boxes represent the percentage of subjects with IgG titers above the cut off value, calculated at the 95^{th} percentile of the control population. Statistical significance: *p<0.001 vs controls. B, IgG titers against human recombinant IFI16 in patients with lc-SSc (57), dc-SSc (25), and healthy controls (80). Graphical representation and statistical analysis as in panel A. Statistical significance: * p=0.02 vs dc-SSc and controls.

FIG. 3. A, Schematic representation of IFI16 and a series of recombinant IFI16 fragments. The two HIN200 domains (domain a and domain b) are indicated. Recombinant peptides corresponding to IFI16 aa 1-204 (IFI16 N-term) and aa 525-726 (IFI16 C-term) are indicated by a box. B, Reactivity to full length IFI16 by immunoblotting. Recombinant IFI16 (IFI16) or control peptide (pET30a) were separated by SDS-PAGE, transferred to nitrocellulose membranes and then incubated with 1:100 dilution of patients sera that were positive (lanes 1-12, with decreasing titers) or negative (lanes 13-14) using ELISA. C, Reactivity to IFI16 N-terminal (IFI16 N-term), C-terminal (IFI16 C-term) fragments or control peptide (pET30a). Nitrocellulose membranes with transferred recombinant proteins were incubated with patients sera that were reactive using immunoblotting to full length IFI16.

FIG. 4. IgG titers against human recombinant IFI16 in patients with multiple sclerosis (MS, N=163) and healthy individuals (CTRL, N=64) as normal controls examined by ELISA with recombinant IFI16 protein. The horizontal bar represents the median.

The present inventors showed, for the first time, enhanced expression levels of the interferon-inducible protein IFI16 in the epidermis and in the dermal inflammatory infiltrate from both SLE and SSc lesions by immunohistochemistry. Additionally, the present inventors confirmed that anti-IFI16 autoantibody titers above the 95^{th} percentile of the controls are significantly elevated in patients with SLE and SjS, but not in those with other autoimmune diseases compared with controls. Interestingly, the present inventors found comparable prevalence of these autoantibodies in SSc as well. Furthermore, the association of anti-IFI16 autoantibody levels in SSc patients with a range of clinical and laboratory parameters was assessed by univariate analysis. The results obtained demonstrated that anti-IFI16 autoreactivity was not associated with either disease duration or disease severity, but with the limited cutaneous form of SSc (lc-SSc).

IFI16 expression was, in fact, greatly increased and found to be ubiquitously expressed in all layers of the epidermis and in the dermal inflammatory infiltrate in the lesional skin from both SLE and SSc patients. Patients with SLE, SjS and SSc exhibited significantly (one-way ANOVA p<0.0001) higher anti-IFI16 antibody levels of the IgG isotype than normal controls (SLE p<0.002, SjS p<0.001 and SSc p<0.0005, respectively). Anti-IFI16 titers above the 95^{th} percentile of the controls were observed in 26% of SLE, 50% of SjS and 21% of SSc respectively. By contrast, anti-IFI16 prevalence was 4% in RA, 5% in CU and 10% in HCV patients respectively.

The most striking data came from logistic regression analysis of the three serological autoimmune markers (anti-IFI16, anti-centromere and anti-topoisomerase I autoantibodies), strongly associated with the cutaneous form of SSc in the patients. This analysis showed that all three serological markers investigated were independent predictors of the cutaneous form of scleroderma, and their combination was able to explain 62% of the associated variability. Without being bound to any specific theory, the present inventors believe that IFI16 reactivity in patient with SSc is an important clue to the development of lc-SSc in anti-centromere and anti-topoisomerase I negative patients. In addition, the finding of anti-IFI16 positivity allowed the present inventors to detect lc-SSc patients among the subgroup negative for both anti-centromere and anti-topoisomerase I reactivity, indicating that determination of IFI16 reactivity in patient with SSc is a valuable tool for the differential diagnosis of lc-SSc in the double negative patients.

Prominent IFI16 expression has been seen in vascular endothelial cells and in stratified squamous epithelia such as skin. Its expression is normally restricted to the basal proliferative layer, suggesting a possible role in the control of skin homeostasis. Transduction of IFI16 into the human umbilical vein endothelial cells (HUVEC) by recombinant viruses efficiently suppressed the formation of capillary-like structures in vitro and cell-cycle progression associated with cell death (8). In addition, type I IFN released by plasmacytoid dendritic cells accumulated in cutaneous SLE lesions mediates inflammation and expression of interferon-inducible genes.

The present invention shows, then, that IFI16 is expressed to a higher level in SSc and SLE lesions in both epithelial and inflammatory cells, and autoantibody titers against it are significantly elevated in both diseases.

Thus, the disease model the present inventors propose is: i) IFI16 expression in lesional skin is enhanced by local type I IFN production or other pro-inflammatory stimuli; ii) IFI16 release, as a consequence of increased cell death, leads to the breakdown in tolerance to this self-antigen as confirmed by the generation of specific anti-IFI16 autoantibodies; iii) an additional pathogenic role of IFI16 is suggested by the observation that its endothelial expression triggers apoptosis, up-regulates the expression of genes encoding adhesion molecules (ICAM-1, E-selectin) and chemokines (IL-8, MCP-1) (S.L., unpublished data) and efficiently suppresses formation of capillary-like structures in vitro.

Finally, although the root causes of these autoimmune diseases are not yet known, with this disease model the present inventors contribute, and advance, the understanding of the cell and molecular mechanisms that impact on, and underlie, SSc and SLE. Moreover, antibody titer against IFI16 protein is an important serologic marker for the laboratory testing for the differential diagnosis of the limited cutaneous form of SSc.

Materials and Methods

Patients and Controls

290 patients were included in this study, classified as follows: 100 SLE, 20 primary SjS, 82 SSc, RA, 38 CU and 163 MS. Sera from 80 sex and age matched healthy subjects were collected from blood banks and represented the control group. 40 patients with chronic HCV infection (kindly provided by Dr. Mario Pirisi, University of Piemonte Orientale, Novara) were also included as additional controls. SSc and primary SjS sera were from Spedali Civili, Brescia. SLE, RA and CU sera were obtained from Istituto Auxologico Italiano, Milan.

All SSc patients (73 women and 9 men, age 21-80, mean age 57) were classified as lc-SSc or dc-SSc according to the criteria of LeRoy et al (9). Patients were regularly assessed using a published consensus core set of variables (10). Disease severity was assessed using the preliminary Medsger scale (11). The most severe involvement was considered for a global severity score. Disability index was evaluated by Health Assessment Questionnaire (HAQ) (12).

Skin biopsies from 6 patients with SSc and 8 patients with SLE were available for immunohistochemistry. They were all taken for diagnostic purposes in stages of active skin disease. Control biopsies were taken from unaffected skin of patients undergoing surgery.

Informed consent for participating in this study was obtained from all donors.

Recombinant Proteins

The entire coding sequence of the b isoform of human IFI16 (SEQ ID. No.:1, HIStagged IFI16; MHHHHHHSSGLVPRGSGMKETAAAKFERQHMDSPDLGTDDDDKAMADIGSSLMSV KMGKKYKNIVLLKGLEVINDYHFRMVKSLLSNDLKLNLKMREEYDKIQIADLMEE KFRGDAGLGKLIKIFEDIPTLEDLAETLKKEKLKVKGPALSRKRKKEVDATSPAP STSSTVKTEGAEATPGAQKRKKSTKEKAGPKGSKVSEEQTQPPSPAGAGMSTAMG RSPSPKTSLSAPPNTSSTENPKTVAKCQVTPRRNVLQKRPVIVKVLSTTKPFEYE TPEMEKKIMFHATVATQTQFFHVKVLNTSLKEKFNGKKIIIISDYLEYDSLLEVN EESTVSEAGPNQTFEVPNKIINRAKETLKIDILHKQASGNIVYGVFMLHKKTVNQ KTTIYEIQDDRGKMDVVGTGQCHNIPCEEGDKLQLFCFRLRKKNQMSKLISEMHS FIQIKKKTNPRNNDPKSMKLPQEQRQLPYPSEASTTFPESHLRTPQMPPTTPSSS FFTKKSEDTISKMNDFMRMQILKEGSHFPGPFMTSIGPAESHPHTPQMPPSTPSS SFLTTLKPRLKTEPEEVSIEDSAQSDLKEVMVLNATESFVYEPKEQKKMFHATVA TENEVFRVKVFNIDLKEKFTPKKIIAIANYVCRNGFLEVYPFTLVADVNADRNME IPKGLIRSASVTPKINQLCSQTKGSFVNGVFEVHKKNVRGEFTYYEIQDNTGKME VVVHGRLTTINCEEGDKLKLTCFELAPKSGNTGELRSVIHSHIKVIKTRKNKKDI LNPDSSMETSPDFFF) was subcloned from pBluescript in the pET30a expression vector (Novagen, Madison, USA), containing an N-terminal histidine tag. The sequences encoding N-terminal (SEQ ID No.:2, HIStagged-IFI16 N-term, amino acid residues 1-200, MHHHHHHSSGLVPRGSGMKETAAAKFERQHMDSPDLGTDDDDKAMADIGSLMSVK MGKKYKNIVLLKGLEVINDYHFRMVKSLLSNDLKLNLKMREEYDKIQIADLMEEK FRGDAGLGKLIKIFEDIPTLEDLAETLKKEKLKVKGPALSRKRKKEVDATSPAPS TSSTVKTEGAEATPGAQKRKKSTKEKAGPKGSKVSEEQTQPPSPAGAGMSTAMGR SPSPKTSLSAPPNTSSTENPKTVAKCQVTPRRNVL) or C-terminal (IFI16 C-term, residues 525-729, (SEQ ID No.:3 MHHHHHHSSGLVPRGSGMKETAAAKFERQHMDSPDLGTDDDDKAMADIGSMVLNA TESFVYEPKEQKKMFHATVATENEVFRVKVFNIDLKEKFTPKKIIAIANYVCRNG FLEVYPFTLVADVNADRNMEIPKGLIRSASVTPKINQLCSQTKGSFVNGVFEVHK KNVRGEFTYYEIQDNTGKMEVVVHGRLTTINCEEGDKLKLTCFELAPKSGNTGEL RSVIHSHIKVIKTRKNKKDILNPDSSMETSPDFFF) fragments of IFI16 were amplified by PCR and cloned in frame in the pET30a vector. Expression and affinity purification were performed following standard procedures. The purity of the proteins was assessed by Coomassie blue staining following 10% sodium dodecyl sulphate-polyacrylamide gel electrophoresis (SDS-PAGE). As negative controls for ELISA and immunoblotting, the polypeptide encoded by the pET30a empty vector (SEQ ID No.:4; control peptide, MHHHHHHSSGLVPRGSGMKETAAAKFERQHMDSPDLGTDDDDKAMAD IGSEFELRRQACGRTRAPPPPPLRSGC) was expressed and purified with the same protocol.

Immunoblotting and Immunohistology

250 ng of recombinant IFI16 (SEQ ID No.:1), IFI16-N-term (SEQ ID No.:2), IFI16 C-term (SEQ ID No.:3) or control peptide (SEQ ID No.:4) were separated using SDS-PAGE under reducing conditions and transferred to nitrocellulose membranes. The membranes were blocked in 3% Top Block (Fluka, St. Louis, USA) and then incubated with 1:100 dilution of the patients sera. After washing, the membranes were incubated with horse radish peroxidase (HRP)-conjugated rabbit anti-human IgG (DakoCytomation, Glostrup, Denmark). The immunocomplexes were detected by enhanced chemiluminescence (ECL, GE Healthcare) and signals acquired with Versadoc3000 (Bio-Rad Laboratories Inc., Hercules, USA). After stripping, the membranes were reprobed with anti-IFI16 C-terminal rabbit polyclonal antibody (13) or anti-IFI16 N-terminal mouse monoclonal antibody (Santa Cruz Biotechnology, Santa Cruz, USA) to confirm the presence of the recombinant proteins.

Immunohistochemical analysis for IFI16 expression was performed on sections from paraffin-embedded tissues placed on silane-coated glass slides as previously described (6).

Determination of Antibody Titers Towards Human Recombinant IFI16 by ELISA

Polystyrene microwell plates for enzyme-linked immunoabsorbent assay (ELISA) (Nunc-Immuno Maxi-Sorp, Nunc, Roskilde, Denmark) were coated for 16 h at 4° C. with 2 μg/ml of either human recombinant IFI16 (SEQ ID No.:1) or control peptide solubilized in 0.2 ml of phosphate-buffered saline (PBS). After blocking with coating buffer containing 3% bovine serum albumin (BSA) in PBS, patients sera, diluted 1:100, were added in duplicate and incubated for 1 h at 37° C. After washing, HRP-conjugated rabbit anti-human IgG (dilution 1:4800) (DakoCytomation) was added and incubated for 1 h at 37° C. After substrate addition, absorbance was measured at 490 nm using a Bio-Rad microplate reader (Bio-Rad). The results were corrected by subtracting the background reactivity of the reference mixture.

Data Analysis and Statistical Calculations

Statistical analysis was performed by SPSS statistical software (SPSS Inc., Chicago, USA) using the one-way ANOVA with Bonferroni Multiple Comparisons tests. Either Fisher's exact test or chi-square test was used for measuring association. The independent effect of significant variables was assessed using forward conditional logistic regression. Positivity cut-off values were calculated as the 95^{th} percentiles in the control population. With substantial deviation from normality, data was natural log transformed before parametric analysis was performed.

### Results

### Example 1

### Anti-IFI16 Autoantibody Levels by ELISA and Skin IFI16 Immunoreactivity were Elevated in SSc and SLE

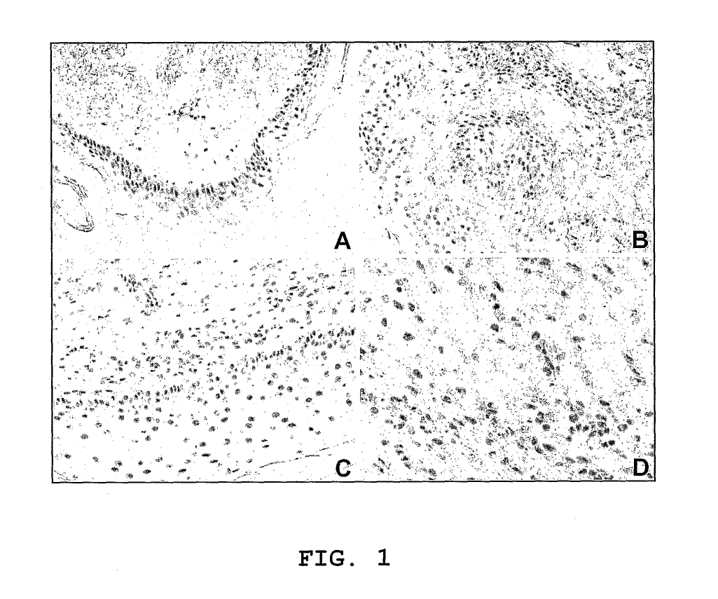

As recently reported in the normal epidermis from healthy controls, IFI16 expression was restricted to the basal layer (FIG. 1A). Notably, as shown in the representative sections in FIG. 1, IFI16 expression was greatly increased and found ubiquitously expressed in all layers of the epidermis in the lesional skin from both SSc (FIG. 1B) and SLE (FIG. 1C) patients. Furthermore, the dermal inflammatory infiltrate showed IFI16 positive staining, indicating that it is expressed to a high level in lymphocytes, fibroblasts and endothelial cells.

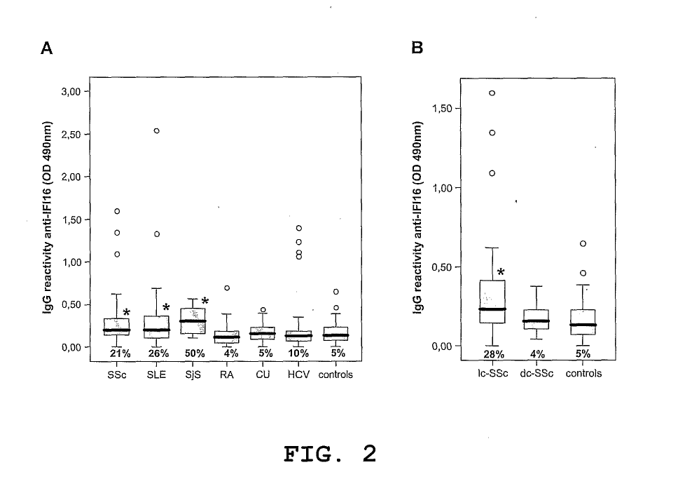

To verify whether the increased expression of IFI16 in the affected skin was related to the presence of anti-IFI16 autoantibodies, their presence and levels in serum samples from 100 SLE patients and 82 SSc patients were assessed using ELISA with recombinant IFI16 protein. Other autoimmune diseases, including SjS, RA and CU as well as healthy individuals as normal controls were also examined. In addition, to further assess the clinical specificity of anti-IFI16 autoantibodies, 40 sera from patients with chronic HCV infection were also subjected to anti-IFI16 ELISA. Absorbance values higher than the 95^{th} percentile of the control population (0.360) were considered to be positive in this study. As depicted in FIG. 2A, patients with SSc, SLE and SjS exhibited significantly (one-way ANOVA p<0.0001) higher anti-IFI16 antibody levels of the IgG isotype than normal controls did (SSc p<0.0005, SLE p<0.002 and SjS p<0.001 respectively). Anti-IFI16 titers above the 95^{th} percentile of the controls were observed in 21% of SSc, 26% of SLE and 50% of SjS respectively. By contrast, anti-IFI16 prevalence was 4% in RA, 5% in CU and 10% in HCV patients respectively. Thus, IgG autoantibody levels were increased in SLE, SjS and SSc but not in other autoimmune diseases, including RA and CU or in patients with increased polyclonal reactivity such as HCV-positive patients.

### Example 2

### Association of anti-IFI16 Antibodies with Clinical Parameters

Since the presence of anti-IFI16 autoantibodies had already been reported in both SjS and SLE, where a significant serological heterogeneity is well known to occur (14), but not in SSc, a decision was taken to gain more insight into the actual role of anti-IFI16 autoantibodies in SSc pathogenesis. Univariate analysis showed that anti-IFI16 autoreactivity in SSc patients was not associated with either disease duration or disease severity as measured by Medsger stage, HAQ disability index, organ involvement and positivity to other autoantibodies (data not shown). By contrast, a strict association between anti-IFI16 reactivity and the cutaneous form of the disease was found, with patients in the limited cutaneous scleroderma (lc-SSc) having higher anti-IFI16 IgG titers than patients with the diffuse form (dc-SSc) (p=0.017). Indeed, as shown in FIG. 2B, anti-IFI16 titers above the 95^{th} percentile of the controls were observed in 28% of patients with lc-SSc but only in 4% of dc-SSc (95% confidence interval for the difference 5-37%).

In line with previous reports, the presence or titers of anti-IFI16 antibodies did not correlate with clinical manifestations or disease activity in both SLE and SjS patients (data not shown).

### Example 3

### Logistic Regression Model and Sensitivity-Specificity Analysis for lc-SSc Prediction

In our samples the cutaneous form of SSc was significantly associated with both anti-centromere (χ²=18.771 p<0.0005) and anti-topoisomerase (χ²=32.689 p<0.0005) autoantibodies. Logistic regression showed that all the three serological markers were independent predictors of the cutaneous form of scleroderma, and their combination was able to explain 62% of the associated variability. This model was able to correctly predict 89% of the clinical presentation forms of scleroderma. Moreover, anti-IFI16 reactivity displayed lower sensitivity (28%) and higher specificity (96%) than those found with either anti-topoisomerase I (95% and 67% respectively) or anti-centromere (65% and 92% respectively). The combined use of anti-IFI16 and anti-centromere markers gave rise to the highest sensitivity and specificity score (79% and 92% respectively). Interestingly, in the SSc subgroup negative for both anti-centromere and anti-topoisomerase I reactivity, all patients with anti-IFI16 positivity displayed the limited cutaneous form of SSc (specificity 100% and positive predictive value 100%).

### Example 4

### Immunoblotting Analysis for anti-IFI16 Antibody

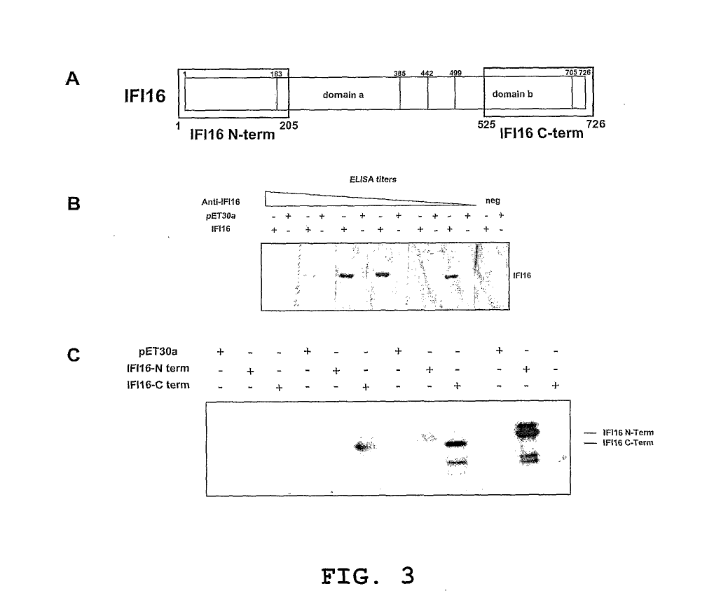

The presence of anti-IFI16 antibodies was also evaluated by immunoblotting analysis using recombinant IFI16 protein either full length or deleted fragments as schematically represented in FIG. 3A. Western blots were performed on 25 SSc sera, 17 from patients with anti-IFI16 titers above and 8 with titers below the 95^{th} percentile of the controls. As illustrated in the representative immunoblotting in FIG. 35, low-titer sera did not exhibit reactivity with IFI16, thus confirming the specificity of the ELISA technique. By contrast, only 10 of the 17 IFI16 high-titer sera were positive, very likely because immunoblot detects antibodies directed against linear epitopes while ELISA either linear and conformational epitopes. Correlation between anti-IFI16 autoantibody titers and the intensity of immunoreactive bands was not depicted.

To further characterize the antigenic specificity of the IFI16 positive sera, the 10 sera recognizing linear epitopes were analyzed for their reactivity against the N-terminal (IFI16 N-term) and C-terminal (IFI16 C-term) fragments of IFI16 respectively. As shown in the representative immunoblot in FIG. 3C, 4 sera displayed reactivity against the N-terminal fragment, 3 against the C-terminal fragment, 2 recognized both fragments and 1 displayed no reaction.

All together these data suggest a polyclonal immune response against IFI16 in patients with SSc.

### Example 5

### Anti-IFI16 Autoantibodies in Patients Affected by Multiple Sclerosis

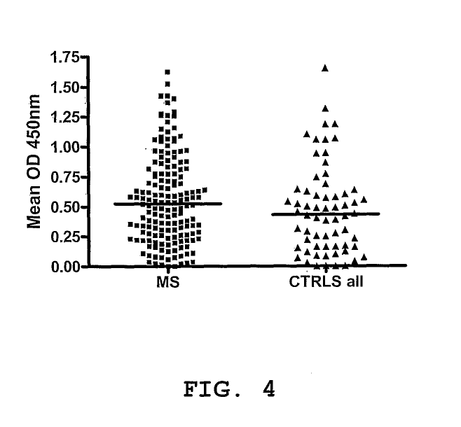

The presence of anti-IFI16 autoantibodies and their levels in serum samples from 163 patients affected by multiple sclerosis (MS) and 64 healthy individuals as normal controls were examined by ELISA with recombinant IFI16 protein. As shown in FIG. 4, patients with MS exhibited significantly (unpaired t-test p<0.05) higher anti-IFI16 antibody levels of the IgG isotype than normal controls did.

Naturally, while the principle of the invention remains the same, the details of construction and the embodiments may widely vary with respect to what has been described and illustrated purely by way of example, without departing from the scope of the present invention as defined in the appended claims.

### REFERENCES

1. Landolfo S, Gariglio M, Gribaudo G, Lembo D. The Ifi 200 genes: an emerging family of IFN-inducible genes. Biochimie 1998; 80 (8-9):721-8.
2. Ludlow L E, Johnstone R W, Clarke C J. The HIN-200 family: more than interferon-inducible genes? Exp Cell Res 2005; 308(1):1-17.
3. Rozzo S J, Allard J D, Choubey D, Vyse T J, Izui S, Peltz G, et al. Evidence for an interferon-inducible gene, Ifi202, in the susceptibility to systemic lupus. Immunity 2001; 15(3):435-43.
4. Seelig H P, Ehrfeld H, Renz M. Interferon-gamma-inducible protein p16. A new target of antinuclear antibodies in patients with systemic lupus erythematosus. Arthritis Rheum 1994; 37(11):1672-83.
5. Uchida K, Akita Y, Matsuo K, Fujiwara S, Nakagawa A, Kazaoka Y, et al. Identification of specific autoantigens in Sjogren's syndrome by SEREX. Immunology 2005; 116(1):53-63.
6. Gariglio M, Azzimonti B, Pagano M, Palestro G, De Andrea M, Valente G, et al. Immunohistochemical expression analysis of the human interferon-inducible gene IFI16, a member of the HIN200 family, not restricted to hematopoietic cells. J Interferon Cytokine Res 2002; 22(7):815-21.
7. Calamia K T, Balabanova M. Vasculitis in systemic lupus erythematosis. Clin Dermatol 2004; 22(2):148-56.
8. Raffaella R, Gioia D, De Andrea M, Cappello P, Giovarelli M, Marconi P, et al. The interferon-inducible IFI16 gene inhibits tube morphogenesis and proliferation of primary, but not HPV16 E6/E7-immortalized human endothelial cells. Exp Cell Res 2004; 293(2):331-45.
9. LeRoy E C, Black C, Fleischmajer R, Jablonska S, Krieg T, Medsger T A, Jr., et al. Scleroderma (systemic sclerosis): classification, subsets and pathogenesis. J Rheumatol 1988; 15(2):202-5.
10. Valentini G, Medsger T A, Jr., Silman A J, Bombardieri S. Conclusion and identification of the core set of variables to be used in clinical investigations. Clin Exp Rheumatol 2003; 21(3 Suppl 29):S47-8.
11. Medsger T A, Jr., Silman A J, Steen V D, Black C M, Akesson A, Bacon P A, et al. A disease severity scale for systemic sclerosis: development and testing. J Rheumatol 1999; 26(10):2159-67.
12. Steen V D, Medsger T A, Jr. The value of the Health Assessment Questionnaire and special patient-generated scales to demonstrate change in systemic sclerosis patients over time. Arthritis Rheum 1997; 40(11):1984-91.
13. Gugliesi F, Mondini M, Ravera R, Robotti A, de Andrea M, Gribaudo G, et al. Up-regulation of the interferon-inducible IFI16 gene by oxidative stress triggers p53 transcriptional activity in endothelial cells. J Leukoc Biol 2005; 77(5):820-9.
14. Sherer Y, Gorstein A, Fritzler M J, Shoenfeld Y. Autoantibody explosion in systemic lupus erythematosus: more than 100 different antibodies found in SLE patients. Semin Arthritis Rheum 2004; 34(2):501-37.

1. (canceled)

2. Method according to claim 4, wherein the reference mixture is represented by a control peptide, particularly peptide SEQ ID NO:4.

3. Method according to claim 4, characterized in that said autoimmunoassay is carried out on whole blood, serum, plasma, saliva, tears, sweat, or synovial fluid.

4. Method of differential diagnosis of limited cutaneous form of scleroderma (lc-SSc) in a subject suspected of or at risk of having an autoimmune disease, wherein autoantibodies against IFI16 protein, fragments or peptides thereof are detected by an ELISA and wherein the results are corrected by subtracting the background reactivity of a reference mixture.

5. Method according to claim 4, characterized in that a difference between the level of said autoantibodies against IFI16 protein fragments or peptides thereof in the subject and a normal level of said autoantibodies is detected.

6. Method according to claim 4, characterized in that the diagnosis is based on the detection of a higher level of autoantibodies against IFI16 protein, fragments or peptides thereof in the subject as compared to the normal level of said autoantibodies.

7. Method according to claim 4, characterized in that the diagnosis is based on the detection of a higher level of autoantibodies against IFI16 protein, fragments or peptides thereof in the subject as compared to a level of said autoantibodies in patients suffering of a diffuse cutaneous form of scleroderma (dc-SSc).

8. Method according to claim 4, characterized in that it comprises the following steps:
a) contacting a sample from a patient with an antigen comprising IFI16 protein, fragments or peptides thereof to form a complex of the autoantibody and the antigen;
b) detecting the presence of the autoantibody antigen complex.

9. Method according to claim 8, characterized in that the sample is selected from the group consisting of whole blood, serum, plasma, saliva, tears, sweat, or synovial fluid.

10. Method according to claim 8, characterized in that the antigen is of human, animal, synthetic or recombinant origin.

11. Method according to claim 8, characterized in that the antigen is bound to a solid carrier.

12. Method according to claim 8, characterized in that the detection is performed using an anti-human IgG antibody.

13. Method according to claim 12, characterized in that the anti-human IgG antibody is conjugated to a detectable marker.

14. Method according to claim 13, characterized in that the detectable marker is selected from horseradish peroxidase, alkaline phosphatase, biotin or fluorescent dyes.

15. Kit for the determination of anti-IFI16 autoantibodies in a sample including a plate of wells having bound thereto IFI16 protein, fragments or peptides thereof, and wells having bound thereto a reference mixture and a detection reagent.

16. Kit according to claim 15, characterized in that the detection reagent is an anti-human IgG antibody conjugated to a detectable marker.

17. Kit according to claim 16, characterized in that the detectable marker is selected from horseradish peroxidase, alkaline phosphatase, biotin or fluorescent dyes.

18. Method of differential diagnosis of limited cutaneous form of scleroderma (lc-SSc) in a subject suspected of or at risk of having an autoimmune disease, wherein a combination of autoantibodies against IFI16 protein, fragments or peptides thereof with anti-centromere autoantibodies is detected.

19. Method of differential diagnosis of limited cutaneous form of scleroderma (lc-SSc) in a subject suspected of or at risk of having an autoimmune disease, wherein a combination of autoantibodies against IFI16 protein, fragments or peptides thereof with anti-centromere autoantibodies and anti-topoisomerase I autoantibodies are detected.

20. Method of diagnosis of multiple sclerosis in a subject, wherein autoantibodies against IFI16 protein, fragments or peptides thereof are detected.
